(19) United States
(12) Patent Application Publication
(10) Pub. No.: US 2007/0082871 A1
(43) Pub. Date: Apr. 12, 2007

(54) Antiresorptive mutual salt of raloxifene and bisphosphonic acid

(75) Inventors: Tae Hee Ha, Suwon-si (KR); Won Jeoung Kim, Suwon-si (KR); Sangmin Yun, Seongnam-si (KR); Cheol Kyung Kim, Namyangju-si (KR); Han Kyong Kim, Yongin-si (KR); Kwee-Hyun Suh, Suwon-si (KR); Gwan Sun Lee, Seoul (KR)

Correspondence Address: SUGHRUE MION, PLLC, 2100 PENNSYLVANIA AVENUE, N.W., SUITE 800, WASHINGTON, DC, 20037, US

(73) Assignee: HANMI PHARM. CO., LTD., Kyungki-do 445-910 (KR)

(21) Appl. No.: 10/579,199

(22) PCT Filed: Nov. 15, 2004
(86) PCT No.: PCT/KR04/02954

§ 371 Date: May 12, 2006

(30) Foreign Application Priority Data
Nov. 14, 2003 (KR) 10-2003-0080494

(51) Int. Cl. A61K 31/675; C07F 9/6533

(52) U.S. Cl. 514/79; 546/22; 544/81

(57) ABSTRACT

The mutual salt of raloxifene and bisphosphonic acid exhibits unexpectedly synergistic effects of two components to enhance bone mineral density (BMD), control blood-calcium density, and lower the serum cholesterol level.

### FIELD OF THE INVENTION

The present invention relates to an effective antiresorptive compound, a method for preparing the same and a pharmaceutical composition containing the same as an active ingredient.

### BACKGROUND OF THE INVENTION

Many bisphosphonic acid-based antiresorptive agents, such as Alendronate (Fosamax®, see U.S. Pat. No. 4,621,077), Etidronate, Clodronate, Pamidronate, Tiludronate, Risedronate and Incadronate have been developed for treating bone-related diseases including osteoporosis caused by upsetting the balance between bone degradation and formation. However such agents are known to cause undesirable side effects such as abnormal bone-metabolism, hypocalcemia, oesophagitis and esophageal ulcer.

There have been numerous attempts to afford a synergistic effect and to resolve such problems associated with bisphosphonic acid-based antiresorptives by administering therewith, estrogen, calcitriol, derivative of triarylethylene, phenylindole, benzothiophene or dihydronaphthalene, or raloxifene (see Lindays, et. al., J. Clin. Endocrinol. Metab., 84, 3076-3081 (1999); Wimalawansa, Am. J. Med., 104, 219-226 (1998); International Patent Publication No. WO01/28564; European Patent Publication No. 693,285; J. Clin. Endocrinol. Metab., 87, 985-992 (2002); and International Patent Publication No. WO02/07733). However, estrogen and calcitriol may cause uterine or breast cancers and hypercalcemia, respectively, and there are several problems related with the combinatorial administration of two drugs even in case of side effect-free raloxifene.

Accordingly, the present inventors have endeavored to develop an antiresorptive compound that is free from the above problems, and have found that a mutual salt of raloxifene and bisphosphonic acid enhances the bone mineral density (BMD) with minimal adverse effect.

### SUMMARY OF THE INVENTION

Accordingly, it is a primary object of the present invention to provide a compound which is superior to the conventional antiresorptive agents in enhancing BMD, controlling blood-calcium density, and lowering serum cholesterol level.

It is another object of the present invention to provide a process for preparing such compound.

It is a further object of the present invention to provide a pharmaceutical composition containing such compound.

### BRIEF DESCRIPTION OF THE DRAWINGS

The above and other objects and features of the present invention will become apparent from the following description of the invention, when taken in conjunction with the accompanying drawings, which respectively show:

FIG. 1: Powder X-ray diffraction spectrum of the inventive mutual salt of raloxifene and alendronic acid (pentahydrate);

FIG. 2: Powder X-ray diffraction spectrum of the inventive mutual salt of raloxifene and risedronic acid (trihydrate).

### DETAILED DESCRIPTION OF THE INVENTION

In accordance with one aspect of the present invention, there is provided a mutual salt of raloxifene and bisphosphonic acid of formula (I):

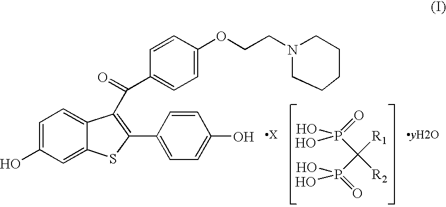

wherein:

R₁ is C₁₋₆ alkyl optionally substituted with one or more substituents selected from the group consisting of NR₃R₄, OH, halogen, C₁₋₆ alkylthio, phenyl, C₃₋₇ cycloalkyl optionally substituted with NR₃R₄ or OH, imidazolyl, pyridyl and imidazopyridyl; C₃₋₆ cycloalkyl optionally substituted with one or more substituents selected from the group consisting of NR₃R₄, OH, halogen, C₁₋₆ alkylthio, phenyl, morpholine and pyridyl; NR₃R₄; halogen; C₁₋₆ alkylthio optionally substituted with one or more substituents selected from the group consisting of NR₃R₄, OH, halogen and phenyl; or phenylthio optionally substituted with one or more substituents selected from the group consisting of halogen, nitro, C₁₋₆ alkyl, C₁₋₆ alkoxy, trifluoromethyl, CONR₃R₄ and CO₂H;

R₂ is hydrogen, OH or halogen;

R₃ and R₄ are each independently hydrogen, C₁₋₆ alkyl or C₃₋₆ cycloalkyl, wherein R₃ and R₄ are optionally fused together with the nitrogen to which they are attached to form a 5 to 7-membered ring;

x is 0.5 or 1; and

y is an integer in the range of 0 to 10.

In the mutual salt of formula (I) of the present invention, preferred R₁ is C₁₋₆ alkyl optionally substituted with one or more substituents selected from the group consisting of NR₃R₄, imidazolyl and pyridyl; NR₃R₄; halogen; or phenylthio substituted with halogen; and y is preferably an integer in the range of 0 to 7.

In the mutual salt of formula (I) of the present invention, representative examples of the bisphosphonic acid part include 1-hydroxyethylidene bisphosphonic acid (etidronic acid), dichloromethylidene bisphosphonic acid (clodronic acid), 3-amino-1-hydroxypropylidene bisphosphonic acid (pamidronic acid), 4-amino-1-hydroxybutylidene bisphosphonic acid (alendronic acid), 4-chlorophenylthiomethylidene bisphosphonic acid (tiludronic acid), 3-(N-methyl-N-n-pentyl)amino-1-hydroxypropylidene bisphosphonic acid (ibandronic acid), 1-hydroxy-2-(3-pyridinyl)ethylidene bisphosphonic acid (risedronic acid), cycloheptylaminomethylidene bisphosphonic acid (incadronic acid), 1-hydroxy-2-(1-imidazolyl)ethylidene bisphosphonic acid (zoledronic acid) and 1-hydroxy-3-(pyrrolidinyl)propylidene bisphosphonic acid; preferably 4-amino-1-hydroxybutylidene bisphosphonic acid (alendronic acid) and 1-hydroxy-2-(3-pyridinyl)ethylidene bisphosphonic acid (risedronic acid).

Representative examples of the compound of formula (I) include mutual salts of raloxifene and etidronic acid (raloxifene 1/2 etidronate 5/2 hydrate), pamidronic acid (raloxifene pamidronate trihydrate), alendronic acid (raloxifene alendronate pentahydrate), risedronic acid (raloxifene risedronate trihydrate), incadronic acid (raloxifene incadronate monohydrate), and zoledronic acid (raloxifene zoledronate tetrahydrate); preferably raloxifene alendronate pentahydrate and raloxifene risedronate trihydrate.

The mutual salt of formula (I) may be polymorphous, or a specific crystal form depending on the state of the hydrate thereof. Therefore, the present invention embraces all crystal forms of the mutual salt of formula (I) within its scope. For example, the raloxifene-alendronate mutual salt in the form of a pentahydrate exhibits characteristic powder X-ray diffraction peaks as shown in Table I.

TABLE I

| 2θ (±0.2) | d | I/Iₒ |
|---|---|---|
| 4.2 | 21.0 | 396 |
| 8.4 | 10.5 | 1000 |
| 9.4 | 9.4 | 397 |
| 9.7 | 9.1 | 900 |
| 10.8 | 8.2 | 247 |
| 13.3 | 6.6 | 281 |
| 13.8 | 6.4 | 406 |
| 16.7 | 5.3 | 265 |
| 18.3 | 4.8 | 251 |
| 18.6 | 4.8 | 443 |
| 19.4 | 4.6 | 421 |
| 19.8 | 4.5 | 581 |
| 20.5 | 4.3 | 308 |
| 20.8 | 4.2 | 349 |
| 21.2 | 4.2 | 354 |
| 21.6 | 4.1 | 562 |
| 25.5 | 3.5 | 225 |
| 26.9 | 3.3 | 238 |

Also, the raloxifene-risedronate mutual salt in the form of a trihydrate exhibits the characteristic powder X-ray diffraction peak pattern showing peaks at diffraction angle listed in Table II.

TABLE II

| 2 (±0.2) | d | I/Iₒ |
|---|---|---|
| 6.8 | 13.0 | 351 |
| 10.3 | 8.6 | 835 |
| 12.3 | 7.2 | 251 |
| 12.9 | 6.8 | 943 |
| 15.2 | 5.8 | 298 |
| 16.5 | 5.4 | 1000 |
| 17.0 | 5.2 | 278 |
| 17.3 | 5.1 | 264 |
| 17.7 | 5.0 | 744 |
| 20.3 | 4.4 | 756 |
| 20.9 | 4.2 | 786 |
| 21.2 | 4.2 | 643 |
| 26.0 | 3.4 | 666 |
| 26.7 | 3.3 | 539 |
| 28.8 | 3.1 | 564 |
| 29.6 | 3.0 | 347 |
| 31.0 | 2.9 | 896 |

The mutual salt of formula (I) may be prepared by reacting a compound of formula (II) or its solvate with a compound of formula (III) or its solvate, in a solvent.

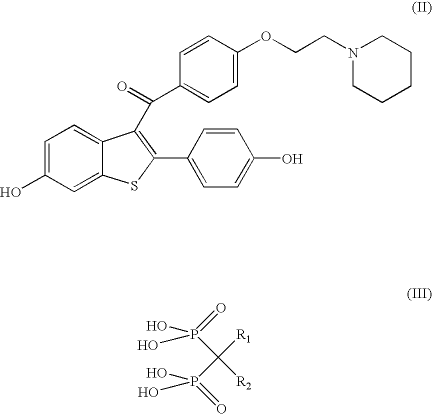

wherein, R₁ and R₂ have the same meanings as defined above.

The solvent employed in the present invention may be selected from the group consisting of water, methanol, ethanol, propanol, isopropanol, acetone, tetrahydrofuran, 1,4-dioxane, acetonitrile, N,N-dimethylformamide, and a mixture thereof; preferably water, and a mixture of water and organic solvent such as acetone, methanol and ethanol.

The compound of formula (III) may be employed in an amount ranging from 1 to 1.5 equivalents, preferably from 1 to 1.1 equivalents based on 1 equivalent of the compound of formula (II), and the reaction may be conducted at a temperature ranging from room temperature to the boiling point of the solvent used for 0.5 to 24 hours, preferably for 2 to 12 hours. After terminating the reaction, the resulting mixture may be cooled to the temperature ranging from 0° C. to room temperature, and filtrated to obtain a solid.

The solid obtained may be filtrated under a reduced pressure, or washed with the same solvent as used in the reaction, and dried at 40° C. to 70° C. under an atmosphere pressure or a reduced pressure.

The compound of formula (II) may be prepared according to the methods described in J. Med. Chem., 27, 1057-1066(1986); U.S. Pat. Nos. 4,418,068 and 5,750,688; and International Publication Nos. WO96/09045, WO97/34888, WO98/49156 and WO01/233069; and the compound of formula (III) may be prepared according to the methods described in U.S. Pat. Nos. 3,366,675; 3,404,178; 4,327,039; 4,621,077; 4,876,248; 4,927,814; 4,970,035; 4,939,130; and 5,583,122.

The mutual salt of formula (I) prepared by the inventive method effectively enhances BMD, controls blood-calcium density, and lowers serum cholesterol level.

Consequently, the present invention also encompasses within its scope a pharmaceutical composition comprising the mutual salt of formula (I) as an active ingredient together with pharmaceutically acceptable carriers, diluent or excipients, for preventing or treating osteoporosis, hypercalcemia and hyperlipidemia.

The pharmaceutical compositions of the present invention may be formulated for oral administration, and the inventive composition for oral administration may take various forms such as solution, emulsions, tablets, coated tablets, powder, rigid or soft capsules, and aqueous dispersion, which is prepared in a conventional manner (see Remington's Pharmaceutical Sciences, Mack Publishing Company, Easton, Pa., 19th Edition (1995)) together with at least one pharmaceutically acceptable carriers such as excipients (e.g. starch, glucose and mannitol); fillers and extenders (e.g., calcium phosphate and silicate derivative); binding agents (e.g., carboxymethyl cellulose or other cellulose derivatives, gelatin, alginate and polyvinyl pyrrolidone); lubricants (e.g., talc, magnesium or calcium stearate and solid state polyethyleneglycol); disintegrants (e.g., povidone, croscarmellose sodium and crospovidone); and surfactants (e.g., polysorbate, cetyl alcohol and glycerol monostearate).

The composition of the present invention may comprise the mutual salt of formula (I) in an amount ranging from 0.1 to 95% by the weight, preferably from 1 to 70% by the weight.

A proper daily dosage of the mutual salt of formula (I) as an active ingredient for a mammal including human ranges from 0.1 to 1,000 mg/kg body weight, preferably from 1 to 250 mg/kg body weight in the oral administration. However, it should be understood that the amount of the active ingredient actually administered should be determined in light of various relevant factors including the condition to be treated, the chosen route of administration, the age and weight of the individual patient, and the severity of the patient's symptoms; and, therefore, the dosage suggested above should not be construed to limit the scope of the invention in any way.

The following Preparation and Examples are given for the purpose of illustration only and are not intended to limit the scope of the invention.

### PREPARATION 1

### Synthesis of 6-hydroxy-2-(4-hydroxyphenyl)benzo[b]thien-3-yl 4-[2-(1-piperidinyl)ethoxy]phenyl methanone (Free Base of Raloxifene)

100 g of 6-hydroxy-2-(4-hydroxyphenyl)benzo[b]thien-3-yl 4-[2-(1-piperidinyl)ethoxy]phenyl methanone hydrochloride (raloxifene hydrochloride) prepared according to the method described in J. Med. Chem., 27, 1057-1066(1986) was added to a mixture of 700 ml of isopropanol and 200 ml of water, 200 ml of 1 N NaOH was added slowly thereto until the pH of the solution became 9.0, and the mixture was stirred at room temperature for 5 hours. The resulting solid was isolated by filtration, washed with a mixture of 100 ml of isopropanol and 100 ml of water, washed with 200 ml of water, and dried at 60° C. to obtain 96 g of the title compound as a yellow isopropanol solvate (0.5 equivalent per mole of the title compound).

Melting point (M.P.) 125˜130° C. ¹H-NMR (DMSO-d₆, ppm): δ 9.75 (bs, 2H), 7.63 (d, 2H), 7.32 (s, 1H), 7.23 (d, 1H), 7.15 (d, 2H), 6.90 (d, 2H), 6.84 (d, 1H), 6.66 (d, 2H), 4.05 (t, 2H), 2.59 (t, 2H), 2.35 (m, 4H), 1.43 (m, 4H), 1.33 (m, 2H).

### PREPARATION 2

### Synthesis of Alendronic Acid

10 g of sodium alendronate trihydrate (30.8 mmol) was added to 120 ml of water, heated to 45-50° C., and 3 ml of concentrated HCl was added thereto. When the reaction mixture turned into a solid suspension, 120 ml of ethanol was added thereto, and stirred at room temperature for 2 hours. The resulting solid was isolated by filtration, washed with ethanol, and dried at 50° C. to obtain 7.5 g of the title compound as a white monohydrate.

M.P. 238˜241° C. ¹H-NMR (DMSO-d₆, ppm): δ 2.95 (t, 2H), 1.94 (m, 4H).

The procedure of Preparation 2, or the procedure described in U.S. Pat. Nos. 3,366,675; 3,404,178; 4,327,039; 4,621,077; 4,876,248; 4,927,814; 4,970,035; 4,939,130; or 5,583,122 was carried out using suitable starting materials to obtain other bisphosphonic acids, or solvates thereof.

### EXAMPLE 1

### Preparation of the Mutual Salt of Raloxifene and Alendronic Acid

5.0 g of 6-hydroxy-2-(4-hydroxyphenyl)benzo[b]thien-3-yl 4-[2-(1-piperidinyl)ethoxy]phenyl methanone (raloxifene) was added to a mixture of 75 ml of ethanol and 75 ml of water, 3.2 g of alendronic acid was added thereto, and the mixture was stirred at room temperature for 24 hours. The resulting mutual salt was isolated by filtration, washed with a mixture of 75 ml of ethanol and 75 ml of water, and dried at 40° C. to obtain 6.5 g of the title compound as a cream-colored pentahydrate.

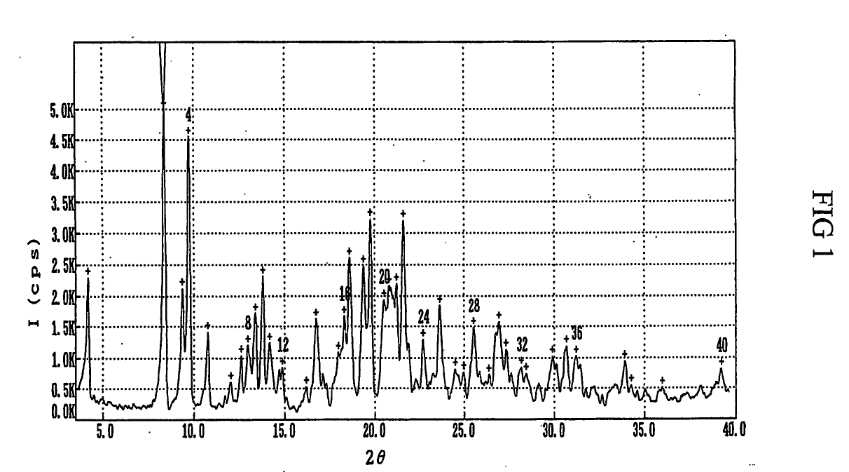

A powder X-ray differaction spectrum of the compound thus obtained is shown in FIG. 1.

M.P. 230° C. (decomposition, dehydration at 130° C.)

Differential scanning calorimetry (DSC): endothermic reaction at 96.1° C., 128.2° C. and 225° C.

Moisture content (Karl-Fisher titrator) 10.9% (theoretical value: 11.1%)

Loss of dry weight (LOD) 11.1%

¹H-NMR (D₂O, ppm): δ 7.63 (t, 3H), 7.26 (s, 1H), 6.72 (m, 3H), 6.41 (d, 2H), 6.32 (d, 2H), 3.97 (bs, 2H), 3.26 (bs, 4H), 2.94 (bs, 2H), 2.74 (t, 2H), 1.91 (m, 4H), 1.62˜1.58 (m, 5H), 1.31 (m, 1H). IR (KBr, cm⁻¹): 3183.4, 2954.3, 2768.9, 1597.8, 1534.8, 1468.9, 1431.6, 1366.2, 1251.7, 1168.7, 1075.3, 1040.4, 907.5, 838.2, 829.6, 529.9.

### EXAMPLE 2

### Preparation of the Mutual salt of Raloxifene and Pamidronic Acid

Method (2-A)

1.0 g of raloxifene was added to 20 ml of 95% ethanol, 0.6 g of pamidronic acid was added thereto, and the mixture was stirred at room temperature for 12 hours. The resulting mutual salt was isolated by filtration, washed with 95% ethanol, and dried at 40° C. for 12 hours to obtain 1.5 g of the title compound as a cream-colored trihydrate.

M.P. 230° C.

Moisture content (Karl-Fisher titrator) 7.5% (theoretical value: 7.1%)

¹H-NMR (D₂O, ppm): δ 7.36 (t, 3H), 7.18 (s, 1H), 6.85 (t, 3H), 6.54 (d, 2H), 6.43 (d, 2H), 4.09 (bs, 2H), 3.34 (bs, 5H), 2.82 (t, 2H), 2.24 (m, 2H), 1.82˜1.63 (m, 5H), 1.38 (m, 1H); IR (KBr, cm⁻¹): 3226.0, 2951.9, 1645.6, 1599.2, 1468.7, 1423.3, 1252.7, 1168.9, 1063.5, 1040.4, 907.8, 835.8, 808.4, 530.3.

Method (2-B)

The procedure of Method (2-A) was repeated except for using a mixture of 10 ml of 2-propanol and 2 ml of water instead of 95% ethanol to obtain 1.1 g of the title compound as a cream-colored solid.

Moisture content (Karl-Fisher titrator) 7.4%

M.P. and ¹H-NMR data were identical with the results of Method (2-A).

Method (2-C)

The procedure of Method (2-A) was repeated except for using 15 ml of water instead of 95% ethanol to obtain 0.98 g of the title compound as a cream-colored solid.

Moisture content (Karl-Fisher titrator) 7.5%

M.P. and ¹H-NMR data were identical with the results of Method (2-A).

### EXAMPLE 3

### Preparation of the Mutual Salt of Raloxifene and Risedronic Acid

Method (3-A)

1.0 g of raloxifene was added to 20 ml of water, 0.72 g of risedronic acid was added thereto, and the mixture was stirred at room temperature for 24 hours. The resulting mutual salt was isolated by filtration, washed with water, and dried at 40° C. for 12 hours to obtain 0.8 g of the title compound as a cream-colored trihydrate.

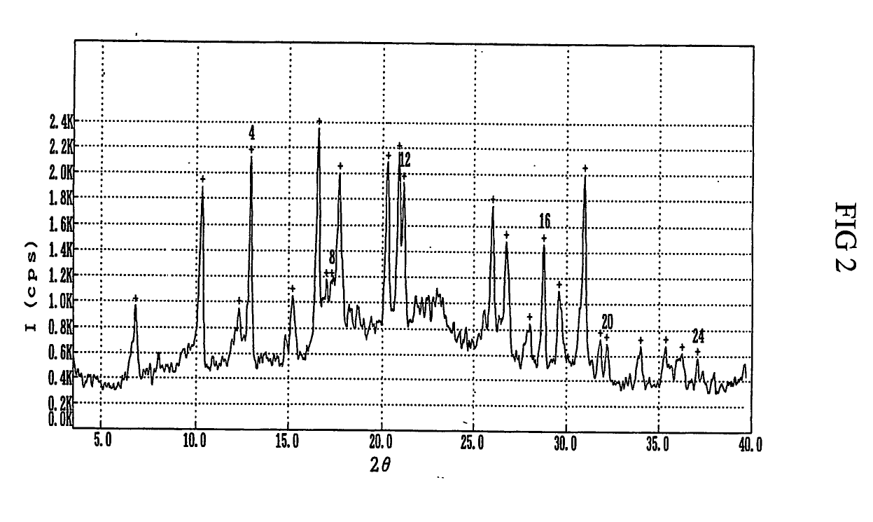

The powder X-ray diffraction spectrum of the compound thus obtained is shown in FIG. 2.

M.P. 230° C.

Moisture content (Karl-Fisher titrator) 6.8% (theoretical value: 6.7%)

¹H-NMR (D₂O, ppm): δ 8.67 (s, 1H), 8.51 (d, 2H), 7.87 (t, 1H), 7.49 (d, 2H), 7.44 (d, 2H), 7.28 (s, 1H), 7.00 (d, 2H), 6.87 (d, 1H), 6.67 (d, 2H), 6.53 (d, 2H), 4.19 (t, 2H), 3.38 (m, 5H), 2.87 (t, 2H), 1.83 (m, 2H), 1.74 (m, 3H), 1.35 (m,1H), IR (KBr, cm⁻¹): 2949.6, 1600.5, 1466.7, 1422.3, 1354.4, 1253.9, 1169.1, 1040.6, 907.7, 834.2, 767.3, 535.0.

Method (3-B)

The procedure of Method (3-A) was repeated except for using a mixture of 15 ml of water and 15 ml of ethanol instead of water to obtain 0.90 g of the title compound as a cream-colored trihydrate.

M.P. and ¹H-NMR data were identical with the results of Method (3-A).

### EXAMPLE 4

### Preparation of the Mutual Salt of Raloxifene and Hemietidronic Acid

5 g of raloxifene was added to a mixture of 50 ml of ethanol and 50 ml of water, 2.47 g of etidronic acid was added thereto, and the mixture was stirred at room temperature for 24 hours. The resulting mutual salt was isolated by filtration, washed with a mixture of 50 ml of ethanol and 50 ml of water, and dried at 40° C. for 12 hours to obtain 5.0 g of the title compound as a cream-colored 5/2hydrate.

M.P. 220° C. (decomposition, dehydration at 170° C.)

Moisture content (Karl-Fisher titrator) 7.5% (theoretical value: 7.25%) ¹H-NMR (DMSO-d₆, ppm): δ 7.84 (d, 2H), 7.52 (s, 1H), 7.43 (d, 1H), 7.34 (d, 2H), 7.11 (d, 2H), 7.03 (d, 2H), 6.85 (d, 2H), 4.42 (t, 2H), 3.20 (m, 2H), 2.96 (bs, 4H), 2.96 (bs, 2H), 1.78 (m, 4H), 1.54 (m, 2H), 1.47 (t, 1.5H).

### EXAMPLE 5

### Preparation of the Mutual Salt of Raloxifene and Zoledronic Acid

2.0 g of raloxifene was added to a mixture of 15 ml of water and 15 ml of ethanol, 1.0 g of zoledronic acid was added thereto, and the mixture was stirred at room temperature for 24 hours. The resulting mutual salt was isolated by filtration, washed with a mixture of 15 ml of water and 15 ml of ethanol, dried at 40° C. to obtain 2.4 g of the title compound as a cream-colored tetrahydrate.

M.P. 235° C.

Moisture content (Karl-Fisher titrator) 8.5% (theoretical value: 8.81%)

¹H-NMR (D₂O, ppm): δ 8.62 (s, H), 7.40 (s, 1H), 7.27 (s, 1H), 7.21 (m, 3H), 7.02 (s, 1H), 6.69 (dd, 4H), 6.32 (dd, 4H), 4.59 (t, 2H), 3.95 (t, 2H), 3.24 (m, 4H), 2.72 (m, 2H).

### EXAMPLE 6

### Preparation of the Mutual Salt of Raloxifene and Incadronic Acid

1.0 g of raloxifene free base was added to 25 ml of 95% ethanol, 0.73 g of incadronic acid was added thereto, and the mixture was stirred at room temperature for 24 hours. The resulting mutual salt was isolated by filtration, washed with 95% ethanol, and dried at 40° C. to obtain 1.2 g of the title compound as a cream-colored monohydrate.

M.P. 225° C.

Moisture content (Karl-Fisher titrator) 2.4% (theoretical value: 2.3%)

¹H-NMR (DMSO-d₆, ppm): δ 7.69 (d, 2H), 7.35 (s, 1H), 7.25 (d, 1H), 7.17 (d, 2H), 6.98 (d, 2H), 6.86 (d, 1H), 6.68 (d, 2H), 4.38 (t, 2H), 3.20 (m, 4H), 2.12 (m, 2H), 2.73 (m, 10H), 1.45 (m, 9H), 1.33 (m, 3H).

### FORMULATION EXAMPLE 1

### Formulation of Soft or Hard Capsule

A soft or hard capsule was prepared using the ingredients listed in Table III according to the conventional method.

TABLE III

| Ingredient | Quantity(mg/capsule) |
|---|---|
| Raloxifene alendronate pentahydrate | 30 |
| Lactose | 215 |
| Magnesium stearate | 2 |
| Colloidal silicon dioxide | 3 |
| Tatal quantity | 250 |

### FORMULATION EXAMPLE 2

### Formulation of Tablet

A tablet was prepared using the ingredients listed in Table IV according to the conventional method.

TABLE IV

| Ingredient | Quantity(mg/tablet) |
|---|---|
| Raloxifene alendronate pentahydrate | 30 |
| Lactose | 185 |
| Povidone | 10 |
| Croscarmellose sodium | 5 |
| Polysorbate | 2 |
| Crospovidone | 15 |
| Magnesium stearate | 1 |
| Solid state polyethyleneglycol | 1 |
| Sodium laurylsulfate | 1 |
| Total quantity | 250 |

### FORMULATION EXAMPLE 3

### Formulation of Suspension

A suspension was prepared using the ingredients listed in Table V according to the conventional method.

TABLE V

| Ingredient | Quantity(mg/volume) | |
|---|---|---|
| Raloxifene alendronate pentahydrate | 100 | |
| Sodium carboxymethylcellulose | 50 | |
| Syrup | 2 | |
| Flavor | Proper quantity | |
| Coloring | Proper quantity | |
| Total volume | 5 | ml |

### TEST EXAMPLE 1

### In Vivo Antiresorptive Activity

In order to investigate the effects of the inventive mutual salts on BMD, bone stiffness, serum cholesterol level and calcium density, in vivo tests were carried out as follows.

Raloxifene alendonate pentahydrate prepared in Example 1, raloxifene hydrochloride, and alendronate (sodium alendronate trihydrate) were each diluted with 1.5% carboxymethylcellulose, and orally administered once a day for 8 weeks to 7-week-old female rats (Sprague-Dawley) that received an operative removal of the ovary. Also, 1.5% carboxymethylcellulose alone was administrated in a same manner to each of ovary-removed rats (group OVX) and normal rats (control, group Sham). After 8 weeks, a femur sample was obtained from each test groups to measure BMD, maximum load, stiffness, trabecular volume of epiphysis, bone volume of metaphysis, blood cholesterol level and blood calcium density. The results are shown in Table VI and VII.

TABLE VI

| | | Administration | | BMD | | Stiffness | | |
|---|---|---|---|---|---|---|---|---|
| | | | Quantity | BMD | Relativ. | Max. load | Stiff. | Relativ. |
| | | Compound | (mg/kg) | (mg/cm²) | Density | (N) | (N/mm) | Stiff.*⁴ |
| Notectomy | Sham | Control | — | 198.22 ± 11.54 | — | 94.40 ± 8.90 | 117.92 ± 34.8 | — |
| Oophorectomy | OVX | Control | — | 78.01 ± 8.86 | 1.00 | 21.91 ± 3.35 | 29.78 ± 10.5 | 1.00 |
| | T1 | Alendronate | 1.0*¹ | 92.57 ± 5.64 | 1.19 | 19.22 ± 2.14 | 19.02 ± 3.86 | 0.88 |
| | T2 | Raloxifene | 1.9 | 91.09 ± 6.98 | 1.17 | 30.00 ± 3.24 | 33.07 ± 9.55 | 1.37 |
| | | hydrochloride | | | | | | |
| | T3 | Raloxifene | 5.6*² | 93.76 ± 6.22 | 1.20 | 32.09 ± 4.37 | 38.64 ± 15.2 | 1.46 |
| | | hydrochloride | | | | | | |
| | T4 | Raloxifene | 2.9*³ | 99.28 ± 6.25 | 1.27 | 26.50 ± 0.73 | 35.34 ± 11.3 | 1.21 |
| | | alenderonate | | | | | | |
| | | pentahydrate | | | | | | |

| | | Trabecular | | | |
|---|---|---|---|---|---|
| | | volume | | Bone volume | |
| | | Vol. | Relativ | Vol. | Relativ |
| | | (%)*⁵ | Vol | (%)*⁶ | Vol |
| Notectomy | Sham | 29.36 ± 3.31 | — | 98.70 ± 1.02 | — |
| Oophorectomy | OVX | 11.56 ± 3.29 | 1.00 | 28.74 ± 13.46 | 1.00 |
| | T1 | 20.27 ± 7.76 | 1.75 | 56.46 ± 17.93 | 1.96 |
| | T2 | 11.25 ± 2.57 | 0.97 | 75.96 ± 17.61 | 2.64 |
| | T3 | 15.15 ± 4.08 | 1.31 | 65.56 ± 15.33 | 2.28 |
| | T4 | 17.81 ± 2.49 | 1.54 | 68.00 ± 9.00 | 2.37 |

*¹ 1/6∴+0 of a clinical dosage (10 mg/adult, about 60 kg)
*² 1/6∴+0 of a clinical dosage (55.7 mg/adult, about 60 kg)
*³1.0 mg of alendronate and 1.9 mg of raloxifene (mol ratio of alendronate:raloxifene = 1:1)
*⁴relative Max. Load value
*⁵(trabecular volume)/(epiphysis volume) × 100(%)
*⁶(bone volume)/(metaphysis volume) × 100(%)

TABLE VII

| | | | | Total blood | | Blood calcium | |
|---|---|---|---|---|---|---|---|
| | | administration)} | | cholesterol | | density | |
| | | | Quantity | Conc. | Relativ. | Conc. | Relativ. |
| | | Compound | (mg/kg) | (mg/dl) | Conc. | (mg/dl) | Conc. |
| Notectomy | Sham | Control | — | 86.26 ± 18.52 | — | 10.00 ± 0.41 | — |
| Oophorectomy | OVX | Control | — | 105.22 ± 23.53 | 1.00 | 9.40 ± 0.20 | 1.00 |
| | T1 | Alendronate | 1.0*¹ | 98.387 ± 17.23 | 0.93 | 6.15 ± 0.39 | 0.65 |
| | T2 | Raloxifene | 1.9 | 41.40 ± 9.71 | 0.39 | 9.20 ± 0.33 | 0.98 |
| | | hydrochloride | | | | | |
| | T3 | Raloxifene | 5.6*² | 40.83 ± 14.2 | 0.39 | 9.42 ± 0.23 | 1.00 |
| | | hydrochloride | | | | | |
| | T4 | Raloxifene | 2.9*³ | 66.86 ± 9.68 | 0.64 | 6.69 ± 0.94 | 0.71 |
| | | alendronate | | | | | |
| | | pentahydrate | | | | | |

*¹1/6∴+0 of a clinical dosage (10 mg/adult, about 60 kg) )}
*²1/6∴+0 of a clinical dosage (55.7 mg/adult, about 60 kg) )}
*³1.0 mg of alendronate and 1.9 mg of raloxifene (mol ratio of alendronate:raloxifene = 1:1)

As shown in Table VI and VII, the inventive mutual salt of raloxifene and alendronic acid markedly enhances BMD, bone stiffness, trabecular volume and bone volume, and also effectively controls the blood cholesterol and calcium level through the synergic effects of its two components, as compared with the individual raloxifene hydrochloride or alendronate.

While the invention has been described with respect to the above specific embodiments, it should be recognized that various modifications and changes may be made to the invention by those skilled in the art which also fall within the scope of the invention as defined by the appended claims.

1-3. (canceled)

4. A crystalline hydrate of raloxifene bisphosphonate, which is selected from the group consisting of crystalline 5/2 hydrate of raloxifene 1/2 etidronate, crystalline trihydrate of raloxifene pamidronate, crystalline pentahydrate of raloxifene alendronate, crystalline trihydrate of raloxifene risedronate, crystalline monohydrate of raloxifene incadronate and crystalline tetrahydrate of raloxifene zoledronate.

5. The crystalline hydrate of claim 4, which is crystalline pentahydrate of raloxifene alendronate.

6. The crystalline hydrate of claim 5, whose powder X-ray diffraction spectrum (I/I₀≧20) shows at 2θ values of 4.2±0.2, 8.4±0.2, 9.4±0.2, 9.7±0.2, 10.8±0.2, 13.3±0.2, 13.8±0.2, 14.2±0.2, 16.7±0.2, 18.3±0.2, 18.6±0.2, 19.4±0.2, 19.8±0.2, 20.5±0.2, 20.8±0.2, 21.2±0.2, 21.6±0.2, 25.5±0.2 and 26.9±0.2.

7. The crystalline hydrate of claim 4, which is crystalline trihydrate of raloxifene risedronate.

8. The crystalline hydrate of claim 7, whose powder X-ray diffraction spectrum (I/I₀≧20) shows at 2θ values of 6.8±0.2, 10.3±0.2, 12.3±0.2, 15.2±0.2, 16.5±0.2, 17.0±0.2, 17.3±0.2, 17.7±0.2, 20.3±0.2, 20.9±0.2, 21.2±0.2, 19.4±0.2, 19.8±0.2, 20.5±0.2, 20.8±0.2, 21.2±0.2, 21.6±0.2, 25.5±0.2 and 26.9±0.2.

9. A process for preparing the crystalline hydrate of raloxifene bisphosphonate of claim 4, which comprises the step of reacting a raloxifene free base or its solvate with a
bisphosphonic acid selected from the group consisting of etidronic acid, pamidronic acid, alendronic acid, risedronic acid, incadronic acid and zoledronic acid, or its solvate, in a solvent.

10. The process of claim 9, wherein the solvent is selected from the group consisting of water, methanol, ethanol, propanol, isopropanol, acetone, tetrahydrofuran, 1,4-dioxane, acetonitrile, N,N-dimethylformamide, and a mixture thereof.

11. A pharmaceutical composition for preventing and treating osteoporosis comprising the crystalline hydrate of raloxifene bisphosphonate of claim 4 as an active ingredient together with pharmaceutically acceptable carriers.

12. A pharmaceutical composition for preventing and treating hypercalcemia comprising the crystalline hydrate of raloxifene bisphosphonate of claim 4 as an active ingredient together with pharmaceutically acceptable carriers.

13. A pharmaceutical composition for preventing and treating hyperlipidemia comprising the crystalline hydrate of raloxifene bisphosphonate of claim 4 as an active ingredient together with pharmaceutically acceptable carriers.
